SEQUENCE LISTING (23 sequences)

SEQ ID NO: 1 - DNA, 21 bp, Artificial Sequence
  Description of Artificial Sequence Synthetic oligonucleotide
nnnnrrrrrrnnnnnnnnnnn

SEQ ID NO: 2 - DNA, 16 bp, Artificial Sequence
  Description of Artificial Sequence Synthetic oligonucleotide
nnnnrrrrrrnnnnnn

SEQ ID NO: 3 - protein, 6 aa, Artificial Sequence
  Description of Artificial Sequence Synthetic 6xHis tag
HHHHHH

SEQ ID NO: 4 - protein, 8 aa, Artificial Sequence
  Description of Artificial Sequence Synthetic 8xHis tag
HHHHHHHH

SEQ ID NO: 5 - protein, 10 aa, Artificial Sequence
  Description of Artificial Sequence Synthetic 10xHis tag
HHHHHHHHHH

SEQ ID NO: 6 - DNA, 6 bp, Artificial Sequence
  Description of Artificial Sequence Synthetic oligonucleotide
tagnnn

SEQ ID NO: 7 - DNA, 6 bp, Artificial Sequence
  Description of Artificial Sequence Synthetic oligonucleotide
taannn

SEQ ID NO: 8 - DNA, 6 bp, Artificial Sequence
  Description of Artificial Sequence Synthetic oligonucleotide
tgannn

SEQ ID NO: 9 - DNA, 6 bp, Artificial Sequence
  Description of Artificial Sequence Synthetic oligonucleotide
tagnnn

SEQ ID NO: 10 - DNA, 6 bp, Artificial Sequence
  Description of Artificial Sequence Synthetic oligonucleotide
taannn

SEQ ID NO: 11 - DNA, 6 bp, Artificial Sequence
  Description of Artificial Sequence Synthetic oligonucleotide
tgannn

SEQ ID NO: 12 - DNA, 6 bp, Artificial Sequence
  Description of Artificial Sequence Synthetic oligonucleotide
tagnnn

SEQ ID NO: 13 - DNA, 6 bp, Artificial Sequence
  Description of Artificial Sequence Synthetic oligonucleotide
taannn

SEQ ID NO: 14 - DNA, 6 bp, Artificial Sequence
  Description of Artificial Sequence Synthetic oligonucleotide
tgannn

SEQ ID NO: 15 - DNA, 93 bp, Artificial Sequence
  Description of Artificial Sequence Synthetic oligonucleotide
ggtggcgacgactcctggagcccgtcagtatcggcggaattccagctgagcgccggtcgctaccattaccagttggtctggtgtcaaaaataa

SEQ ID NO: 16 - DNA, 6 bp, Artificial Sequence
  Description of Artificial Sequence Synthetic oligonucleotide
tagata

SEQ ID NO: 17 - DNA, 41 bp, Artificial Sequence
  Description of Artificial Sequence Synthetic primer
aaaaactcgagcatcatcatcatcatcattagataggtggc

SEQ ID NO: 18 - DNA, 48 bp, Artificial Sequence
  Description of Artificial Sequence Synthetic primer
aaaaaccatggnnnnnnyyyyyynnnaaacgggtatggagaaacagta

SEQ ID NO: 19 - DNA, 3943 bp, Artificial Sequence
  Description of Artificial Sequence Synthetic polynucleotide
taaacggtctccagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcagatcaattcgcgcgcgaaggcgaagcggcatgcataatgtgcctgtcaaatggacgaagcagggattctgcaaaccctatgctactccgtcaagccgtcaattgtctgattcgttaccaattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagagaagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgttt

SEQ ID NO: 20 - DNA, 828 bp, Artificial Sequence
  Description of Artificial Sequence Synthetic polynucleotide
actgtttctccatacccgtttcaggaggagaggagagatatgagtgtgatcgctaaacaaatgacctacaaggtttatatgtcaggcacggtcaatggacactactttgaggtcgaaggcgatggaaaaggaaagccttacgagggggagcagacggtaaagctcactgtcaccaagggcggacctctgccatttgcttgggatattttatcaccacagtgtcagtacggaagcataccattcaccaagtaccctgaagacatccctgactatgtgaagcagtcattcccggagggctatacatgggagaggatcatgaactttgaagatggtgcagtgtgtactgtcagcaatgattccagcatccaaggcaactgtttcatctaccatgtcaagttctctggtttgaactttcctcccaatggacctgtcatgcagaagaagacacagggctgggaacccaacactgagcgtctcattgcacgagatggaatgctgctaggaaacaactttatggctctgaagttagaaggaggcggtcactatttgtgtgaattcaaaactacttacaaggcaaagaagcctgtggagatgccagggtatcactatgttgaccgcaaactggatgtaaccaatcacaacaaggattacacttcggttgagctgtgtgaaatttccattgcacgcaaacctgtggtcgccctcgagcatcaccaccatcaccattagatagatagcgacgactcctggagcccgtcagtatcggcggaattccagctgagcgccggtcgctaccattaccagttggtctggtgtcaaaaataataa

SEQ ID NO: 21 - DNA, 674 bp, Artificial Sequence
  Description of Artificial Sequence Synthetic polynucleotide
ccatggggagtgtgatcgctaaacaaatgacctacaaggtttatatgtcaggcacggtcaatggacactactttgaggtcgaaggcgatggaaaaggaaagccttacgagggggagcagacggtaaagctcactgtcaccaagggcggacctctgccatttgcttgggatattttatcaccacagtgtcagtacggaagcataccattcaccaagtaccctgaagacatccctgactatgtgaagcagtcattcccggagggctatacatgggagaggatcatgaactttgaagatggtgcagtgtgtactgtcagcaatgattccagcatccaaggcaactgtttcatctaccatgtcaagttctctggtttgaactttcctcccaatggacctgtcatgcagaagaagacacagggctgggaacccaacactgagcgtctcattgcacgagatggaatgctgctaggaaacaactttatggctctgaagttagaaggaggcggtcactatttgtgtgaattcaaaactacttacaaggcaaagaagcctgtggagatgccagggtatcactatgttgaccgcaaactggatgtaaccaatcacaacaaggattacacttcggttgagctgtgtgaaatttccattgcacgcaaacctgtggtcgccctcgag

SEQ ID NO: 22 - DNA, 2971 bp, Artificial Sequence
  Description of Artificial Sequence Synthetic polynucleotide
atgatagatcccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggtaccagaagcggtgccggaaagctggctggagtgcgatcttcctgaggccgatactgtcgtcgtcccctcaaactggcagatgcacggttacgatgcgcccatctacaccaacgtaacctatcccattacggtcaatccgccgtttgttcccacggagaatccgacgggttgttactcgctcacatttaatgttgatgaaagctggctacaggaaggccagacgcgaattatttttgatggcgttaactcggcgtttcatctgtggtgcaacgggcgctgggtcggttacggccaggacagtcgtttgccgtctgaatttgacctgagcgcatttttacgcgccggagaaaaccgcctcgcggtgatggtgctgcgttggagtgacggcagttatctggaagatcaggatatgtggcggatgagcggcattttccgtgacgtctcgttgctgcataaaccgactacacaaatcagcgatttccatgttgccactcgctttaatgatgatttcagccgcgctgtactggaggctgaagttcagatgtgcggcgagttgcgtgactacctacgggtaacagtttctttatggcagggtgaaacgcaggtcgccagcggcaccgcgcctttcggcggtgaaattatcgatgagcgtggtggttatgccgatcgcgtcacactacgtctgaacgtcgaaaacccgaaactgtggagcgccgaaatcccgaatctctatcgtgcggtggttgaactgcacaccgccgacggcacgctgattgaagcagaagcctgcgatgtcggtttccgcgaggtgcggattgaaaatggtctgctgctgctgaacggcaagccgttgctgattcgaggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctaccggcgatgagcgaacgcgtaacgcgaatggtgcagcgcgatcgtaatcacccgagtgtgatcatctggtcgctggggaatgaatcaggccacggcgctaatcacgacgcgctgtatcgctggatcaaatctgtcgatccttcccgcccggtgcagtatgaaggcggcggagccgacaccacggccaccgatattatttgcccgatgtacgcgcgcgtggatgaagaccagcccttcccggctgtgccgaaatggtccatcaaaaaatggctttcgctacctggagagacgcgcccgctgatcctttgcgaatacgcccacgcgatgggtaacagtcttggcggtttcgctaaatactggcaggcgtttcgtcagtatccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccagcgctgacggaagcaaaacaccagcagcagtttttccagttccgtttatccgggcaaaccatcgaagtgaccagcgaatacctgttccgtcatagcgataacgagctcctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccgggcacatcagcgcctggcagcagtggcgtctggcggaaaacctcagtgtgacgctccccgccgcgtcccacgccatcccgcatctgaccaccagcgaaatggatttttgcatcgagctgggtaataagcgttggcaatttaaccgccagtcaggctttctttcacagatgtggattggcgataaaaaacaactgctgacgccgctgcgcgatcagttcacccgtgcaccgctggataacgacattggcgtaagtgaagcgacccgcattgaccctaacgcctgggtcgaacgctggaaggcggcgggccattaccaggccgaagcagcgttgttgcagtgcacggcagatacacttgctgatgcggtgctgattacgaccgctcacgcgtggcagcatcaggggaaaaccttatttatcagccggaaaacctaccggattgatggtagtggtcaaatggcgattaccgttgatgttgaagtggcgagcgatacaccgcatccggcgcggattggcctgaactgccagctggcgcaggtagcagagcgggtaaactggctcggattagggccgcaagaaaactatcccgaccgccttactgccgcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagtcaacagcaactgatggaaaccagccatcgccatctgctgcacgcggaagaaggcacatggctgaatatcgacggtttccatatggggattggtg

SEQ ID NO: 23 - DNA, 6 bp, Artificial Sequence
  Description of Artificial Sequence Synthetic oligonucleotide
tagtat

(19) United States
(12) Patent Application Publication
(10) Pub. No.: US 2015/0267209 A1
(43) Pub. Date: Sep. 24, 2015

(54) System and Method for Visualization of Optimized Protein Expression

(71) Applicant: Life Technologies Corporation, Carlsbad, CA (US)

(72) Inventors: Jeffrey Rogers, Escondido, CA (US); Julia Fletcher, Carlsbad, CA (US); Kevin Lowitz, Poway, CA (US)

(21) Appl. No.: 14/437,317

(22) PCT Filed: Oct. 22, 2013
(86) PCT No.: PCT/US2013/066204

§ 371 Date: Apr. 21, 2015

Related U.S. Application Data
(60) Provisional application No. 61/716,951, filed on Oct. 22, 2012.

(51) Int. Cl. C12N 15/67; C12N 9/38; C07K 2/00; C12P 21/00; C12N 15/70

(52) U.S. Cl. CPC C12N 15/67; C12P 21/00; C12N 15/70; C07K 2/00; C12N 9/2471; C07K 2319/60; C07K 2319/40

(57) ABSTRACT

The present disclosure describes a novel recombinant gene expression system and a method of its use. The recombinant gene expression system is adapted to enable a user to monitor the successful expression of a protein in real time by allowing a small portion (e.g., less than 10%) of the target protein to be expressed as a fusion with a readily detectable reporter. The recombinant gene expression system further allows a user to select a clone that exhibits high, medium or low expression by providing a random ribosomal binding site having the nucleotide sequence N₄R₆N_{X}, where N is A, T, G, or C, where R is A or G, and where x is an integer from 6 to 11 (SEQ ID NO: 1) upstream of the cloned gene. By selecting a clone exhibiting a desired relative level of reporter gene expression, a user can tailor the expression level of the desired protein.

### SEQUENCE LISTING

The instant application contains a Sequence Listing which has been submitted electronically in ASCII format and is hereby incorporated by reference in its entirety. Said ASCII copy, created on Dec. 13, 2013, is named LT00712PCT_SL.txt and is 19,940 bytes in size.

### TECHNICAL FIELD OF THE INVENTION

The present invention generally relates to the field of recombinant protein expression in host cells. More particularly, the invention relates to an expression cloning vector system and method of use that allows a user to select expression clones having a desired level of protein expression. The invention further relates to an expression cloning vector system and method that allows a user to readily detect protein expression in cells in real time.

### BACKGROUND OF THE INVENTION

The general principle of protein expression in cultured bacterial or eukaryotic cells or in whole animals by various gene transfer methods is well known and a key strategy in both basic research and biotechnological applications. For large-scale protein production and for many small-scale applications, efficient transient expression of a recombinant product is an absolute requirement. However, difficulties exist in identifying host cells that produce recombinant protein at sufficient levels and in identifying host cell clones that maintain expression levels during prolonged cultivation periods. Additionally, variability in the expression efficiencies of specific clones makes identifying and selecting clones with tailored levels of protein expression challenging.

The process of generating a clone that is optimized for optimized recombinant protein expression in cells can take several weeks or even months. Merely cloning a cDNA into an expression vector is no guarantee that desired protein will be expressed in host cells at a desired level, if at all. Currently, methods to visualize protein expression in E. coli in real-time require generating a fusion protein or expressing a reporter protein, and these methods come without options to select the clone that exhibits optimized expression characteristics.

There exists a need for an expression vector system that allows a user to select a clone that expresses a protein at a desired level. There also exists a need for an expression vector system that allows a user to determine whether a specific clone expresses a protein in real-time, without having to conduct time-consuming analysis and/or modification of the expressed clone.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The above brief description as well as further objects, features and advantages of the methods and apparatus of the present invention will be more fully appreciated by reference to the following detailed description of presently preferred but nonetheless illustrative embodiments in accordance with the present invention when taken in conjunction with the accompanying drawings:

FIG. 1 is a schematic depiction of an expression vector system according to some preferred though non-limiting embodiments (figure discloses SEQ ID NOS 15, 2, 3, 6, and 2, left to right, top to bottom, respectively, in order of appearance); and

FIG. 2 describes a typical method for producing a recombinant using an expression vector system according to some preferred though non-limiting embodiments (figure discloses SEQ ID NOS 2, 3, and 16, respectively, in order of appearance). The detailed methods and procedure employed to prepare and to use the expression vector system are set forth in the EXAMPLES section below.

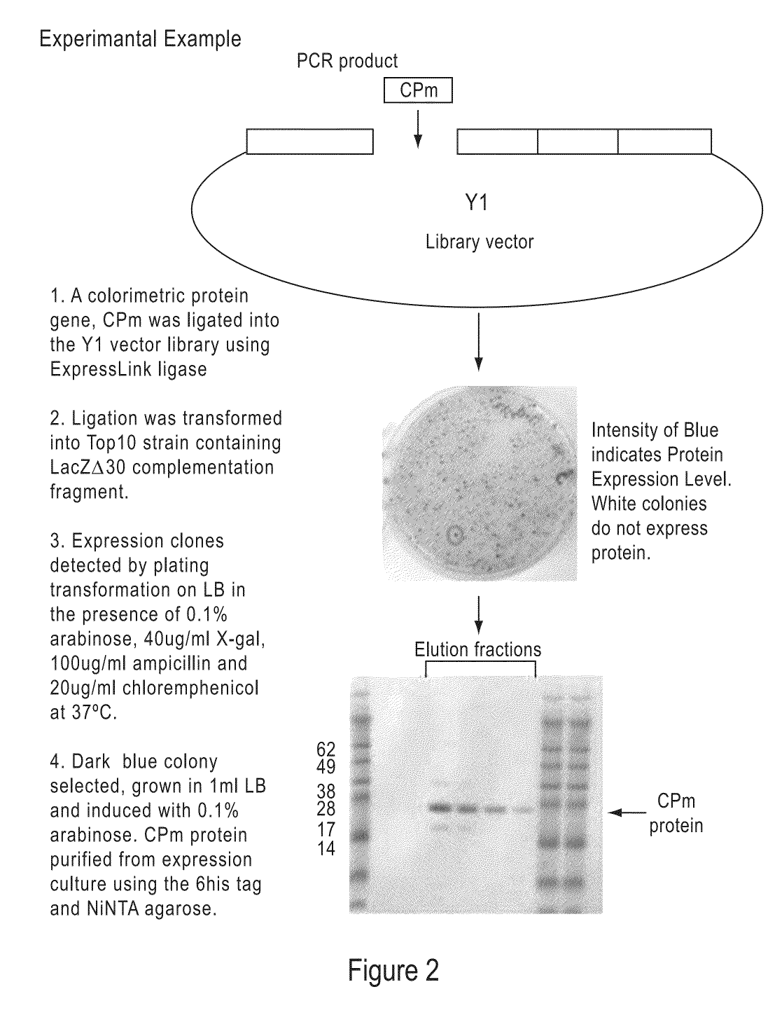

### SUMMARY AND DETAILED DESCRIPTION OF THE INVENTION

Unless defined otherwise, all technical and scientific terms used herein have the same meaning as is commonly understood by one of ordinary skill in the art to which these inventions belong. All patents, patent applications, published applications, treatises and other publications referred to herein, both supra and infra, are incorporated by reference in their entirety. If a definition and/or description is explicitly or implicitly set forth herein that is contrary to or otherwise inconsistent with any definition set forth in the patents, patent applications, published applications, and other publications that are herein incorporated by reference, the definition and/or description set forth herein prevails over the definition that is incorporated by reference.

The practice of the disclosure will employ, unless otherwise indicated, conventional techniques of molecular biology, microbiology and recombinant DNA techniques, which are within the skill of the art. Such techniques are explained fully in the literature. See, for example, Sambrook, J., and Russell, D. W., 2001, Molecular Cloning: A Laboratory Manual, Third Edition; Ausubel, F. M., et al., eds., 2002, Short Protocols In Molecular Biology, Fifth Edition.

Note that not all of the activities described in the general description or the examples are required, that a portion of a specific activity may not be required, and that one or more further activities may be performed in addition to those described. Still further, the order in which activities are listed are not necessarily the order in which they are performed.

In some instances, some concepts have been described with reference to specific embodiments. However, one of ordinary skill in the art appreciates that various modifications and changes can be made without departing from the scope of the invention as set forth in the claims below. Accordingly, the specification and figures are to be regarded in an illustrative rather than a restrictive sense, and all such modifications are intended to be included within the scope of invention.

As used herein, the terms “comprising” (and any form or variant of comprising, such as “comprise” and “comprises”), “having” (and any form or variant of having, such as “have” and “has”), “including” (and any form or variant of including, such as “includes” and “include”), or “containing” (and any form or variant of containing, such as “contains” and “contain”), are inclusive or open-ended and do not exclude additional, unrecited additives, components, integers, elements or method steps. For example, a process, method, article, or apparatus that comprises a list of features is not necessarily limited only to those features but may include other features not expressly listed or inherent to such process, method, article, or apparatus.

Unless expressly stated to the contrary, “or” refers to an inclusive- or and not to an exclusive- or. For example, a condition A or B is satisfied by any one of the following: A is true (or present) and B is false (or not present), A is false (or not present) and B is true (or present), and both A and B are true (or present).

Benefits, other advantages, and solutions to problems have been described with regard to specific embodiments. However, such benefits, advantages, solutions to problems, and any feature(s) that may cause any benefit, advantage, or solution to occur or become more pronounced are not to be construed as a critical, required, or essential feature of any or all the claims.

After reading the specification, skilled artisans will appreciate that certain features that are, for clarity, described herein in the context of separate embodiments can also be provided in combination in a single embodiment. Conversely, various features that are, for brevity, described in the context of a single embodiment can also be provided separately or in any subcombination. Further, references to values stated in ranges include each value within that range.

Also, the use of articles such as “a”, “an” or “the” are employed to describe elements and components described herein. This is done merely for convenience and to give a general sense of the scope of the invention. This description should be read to include one or at least one and the singular also includes the plural unless it is obvious that it is meant otherwise. Accordingly, the terms “a,” “an,” and “the” and similar referents used herein are to be construed to cover both the singular and the plural unless their usage in context indicates otherwise. Accordingly, the use of the word “a” or “an” or “the” when used in the claims or specification, including when used in conjunction with the term “comprising”, may mean “one,” but it is also consistent with the meaning of “one or more,” “at least one,” and “one or more than one.”

The terms “modification” or “modified” and their variants, as used herein with reference to a protein comprise any change in the structural, biological and/or chemical properties of the protein. In some embodiments, the modification can include a change in the amino acid sequence of the protein. For example, the modification can optionally include one or more amino acid mutations, including without limitation amino acid additions, deletions and substitutions (including both conservative and non-conservative substitutions).

The term “conservative” and its variants, as used herein with reference to any change in amino acid sequence, refers to an amino acid mutation wherein one or more amino acids is substituted by another amino acid having highly similar properties. For example, one or more amino acids comprising nonpolar or aliphatic side chains (for example, glycine, alanine, valine, leucine, isoleucine or proline) can be substituted for each other. Similarly, one or more amino acids comprising polar, uncharged side chains (for example, serine, threonine, cysteine, methionine, asparagine or glutamine) can be substituted for each other. Similarly, one or more amino acids comprising aromatic side chains (for example, phenylalanine, tyrosine or tryptophan) can be substituted for each other. Similarly, one or more amino acids comprising positively charged side chains (for example, lysine, arginine or histidine) can be substituted for each other. Similarly, one or more amino acids comprising negatively charged side chains (for example, aspartic acid or glutamic acid) can be substituted for each other. In some embodiments, the modified polymerase is a variant that comprises one or more of these conservative amino acid substitutions, or any combination thereof. In some embodiments, conservative substitutions for leucine include: alanine, isoleucine, valine, phenylalanine, tryptophan, methionine, and cysteine. In other embodiments, conservative substitutions for asparagine include: arginine, lysine, aspartate, glutamate, and glutamine.

It is an object of the embodiments described herein to provide an expression vector system that enables a user to select clones that express a protein in host cells at a desired level. The system expression vector system described below is optimized to allow a user to select differentiate the level of protein expression between individual closes, thereby enabling the user to select clones that express the desired protein at high, medium or low levels.

It is a further object of the embodiments described herein to provide an expression vector system that enables a user to readily determine, in real-time, whether a selected clone is expressing a desired protein in culture, without the user having to process a sample or otherwise perform analysis of the expressed protein.

It is yet a further object of the embodiments described herein to provide an expression vector system that perform the above functions, without a user being required to subject the final expressed protein to rigorous downstream purification steps.

It is another object of the embodiments described herein to provide an expression vector system that enables partial suppression of a stop codon in a host cell so that less than about 10%, less than about 9%, les than about 8%, less than about 7%, less than about 6%, less than about 5%, less than about 4%, less than about 3%, less than about 1%, less than about 0.5% or less than about 0.1% of a protein translated from the expression vector is fused to a detectable reporter protein located downstream of the partially suppressed stop codon.

It is a further object of the embodiments described herein to provide methods for using the embodied expression vectors systems to select clones that express a protein at a desired level.

It is a further object of the embodiments described herein to provide methods for using the embodied expression systems to determine whether a protein is being expressed from a cloned gene of interest in real-time, without having to isolate, prepare, process or analyze samples containing the protein of interest.

In some embodiments, an expression vector system may include a vector backbone functionally linked to a promoter capable of driving transcription in a host cell, The expression vector system many include one or more of a random ribosomal binding site (RRBS) functionally linked to and positioned downstream from the promoter, a stop codon context element having a stop codon and at least onel 3-nucleotide codon in frame with the stop codon, and at least a portion of a gene encoding a reporter protein downstream of and in-frame with the stop codon context element.

The expression vector backbone may be any vector backbone familiar to a practitioner having ordinary skill level in the art without limitation. The selection of a suitable vector backbone as well as the functional elements required in such a vector backbone is well known to such a person. In some preferred though non-limiting embodiments, a suitable vector backbone may be a pBAD vector backbone, or any suitable variant thereof.

In some embodiments, an expression vector system may optionally include a gene of interest inserted between the RRBS and the stop codon context element. In other embodiments, the expression vector system may be adapted to allow a user to readily insert a gene of interest therein, optionally between the RRBS and the stop codon context element. The expression vector system of the present embodiments will be particularly adapted such that expression of the gene of interest is promoted under suitable conditions in a host cell. In an embodiment, the gene of interest is in-frame with the stop codon context element and the portion of the gene encoding a reporter protein.

In some embodiments, the gene of interest, the stop codon context element and the portion of the gene encoding the reporter protein may form an open reading frame.

In some embodiments, and expression vector system may be particularly adapted to drive expression of a gene of interest in a host cell under suitable permissive conditions. The host cell may be, in certain embodiments, a eukaryotic cell. Suitable eukaryotic cells may include any eukaryotic cell capable of being cultured and expressing recombinant nucleic acids transferred to the cell interior. Other suitable cells may include cells in tissue in an animal in vivo. Suitable eukaryotic cells may include cultured eukaryotic cells, stable cell lines, primary cells, yeast cells, fungal cells, plant cells and the like, or may include cells in vivo in an organism. In such embodiments, the elements of the vector backbone, including but not limited to the promoter, may be selected such that expression of the gene of interest is optimized in the host cell. In embodiments where protein expression is to be carried out in eukaryotic cells, a eukaryotic promoter may be selected as the promoter use in the expression vector system. In some preferred though non-limiting embodiments, a host cell may a prokaryotic cell. Suitable prokaryotic cells may include any bacterial cell capable of being cultured and expressing recombinant nucleic acids transferred to the cell interior. In such embodiments, the elements of the vector backbone, including but not limited to the promoter, may be selected such that expression of the gene of interest is optimized in the host prokaryotic cell. In embodiments where protein expression is to be carried out in prokaryotic cells, a prokaryotic promoter may be selected as the promoter use in the expression vector system. In particularly preferred though non-limiting embodiments, the cell may be an E. coli cell, such as, e.g., TOP10 or DH1013 cells. In such embodiments, the expression vector may be selected from the list consisting of SP6, T7, T3 and P_{BAD} (araBAD).

In an embodiment, an expression system may include an RRBS that forms all or at least a portion of the 5′-UTR of an mRNA transcript transcribed from the expression vector. The RRBS may generally include the nucleotide sequence N₄R₆N_{X}, where N is A, T, G, or C, where R is A or G, and where X is an integer from 6 to 11 (SEQ ID NO: 1). In some non-limiting embodiments, an expression vector system may include the nucleotide sequence NNNNRRRRRRNNNNNN (SEQ ID NO: 2). In some embodiment, an RRBS may optionally include a translational initiation site. In other embodiments, a translational initiation site may be included in the user-provided gene of interest.

In some embodiments, an expression vector system may optionally include or more cloning sites positioned between the RRBS and the stop codon context element. The cloning sites may be adapted or selected to facilitate the introduction of a user-provided or defined gene of interest into the expression vector. The optional cloning sites will preferably enable a user to introduce the gene of interest so that the gene forms an open reading frame with the stop codon context element and the portion of a gene encoding a reporter protein. In some embodiments, the expression vector system may optionally be adapted for blunt-end cloning of a user-provided or defined cDNA between the RRBS and the stop codon context element. In other optional and non-limiting embodiments, an expression vector system may be adapted for TOPO®-cloning, for GATEWAY® Cloning or for TOPO® GATEWAY® cloning.

In some embodiments, and expression vector system may optionally include a nucleotide sequence encoding a Tag fusion protein. The nucleotide sequence encoding a Tag fusion protein may be in-frame with the stop codon context element and the portion of the gene encoding a reporter protein. Exemplary though non-limiting Tag-fusion proteins contemplated for use with the embodiments disclosed herein may include, though are not limited to, HIS₆ (SEQ ID NO: 3), HIS₈ (SEQ ID NO: 4), HIS₁₀ (SEQ ID NO: 5), MYC, FLAG, T7 Tag, GST, MBP, HA, S-Tag, V5 Epitope, Pel B, Xpress Epitope, NusA, CBP, GFP, Trx, Mistic, Sumo and DSCBc. In certain preferred though non-limiting embodiments, a Tag fusion protein may include one or more of HIS₆ (SEQ ID NO: 3), HIS₈ (SEQ ID NO: 4), HIS₁₀ (SEQ ID NO: 5), MYC, FLAG, GST, MBP and HA. Most preferably, a Tag fusion protein is one or more of HIS₆ (SEQ ID NO: 3), HIS₈ (SEQ ID NO: 4) and HIS₁₀ (SEQ ID NO: 5).

In an embodiment, an expression vector system may include a stop codon context element. The stop codon context element may be selected such that up to about 10% of a protein, up to about 8% of a protein, up to about 5% of a protein, up to about 2% of a protein, up to about 1% of a protein, up to about 0.5% of a protein, up to about 0.2% of a protein, or up to about 0.1% of a protein translated from an mRNA produced in a cell using the expression vector system is expressed as a fusion protein with the reporter protein. In some embodiments, the stop codon context element may be selected such that between about 1% to about 10% of a protein, between about 0.5% to about 5% of a protein, between about 0.05% to about 0.5% of a protein, between about 0.01% to about 0.1% of a protein, or between about 0.01% to about 1% of a protein translated from an mRNA produced in a cell using the expression vector system is expressed as a fusion protein with the reporter protein.

In some embodiments, the nucleotide sequence of a stop codon context element for use in the embodiments described herein may be selected from the list consisting of the nucleotide sequences TAGNNN (SEQ ID NO: 6), TAANNN (SEQ ID NO: 7) or TGANNN (SEQ ID NO: 8), where N is A, T, G or C, and the nucleotide sequence NNN is selected to allow up to about 10%, up to about 5%, up to about 2%, up to about 1%, up to about 0.5%, up to about 0.2%, or up to about 0.1% or between about 1% to about 10%, between about 0.5% to about 5%, between about 0.05% to about 0.5%, between about 0.01% to about 0.1%, or between about 0.01% to about 1% suppression of the stop codon. In certain exemplary though non-limiting embodiments, the nucleotide sequence of a stop codon context element may be selected from the list of nucleotide sequences consisting of TAGNNN (SEQ ID NO: 9), TAANNN (SEQ ID NO: 10) or TGANNN (SEQ ID NO: 11), where N is A, T, G or C, and the nucleotide sequence NNN is selected from the list consisting of GAT, GCT, CGC, GTT, AAT, ACT, GAG, ATA, CAT, CGT, CCT, TAT, TCT, and ATT. In certain preferred though non-limiting embodiments, the nucleotide sequence of a stop codon context element may be selected from the list of nucleotide sequences consisting of TAGNNN (SEQ ID NO: 12), TAANNN (SEQ ID NO: 13) or TGANNN (SEQ ID NO: 14), where N is A, T, G or C, and the nucleotide sequence NNN is selected from either TAT or ATA.

In some embodiments, an expression vector system may include at least a portion of a gene encoding a reporter protein. The reporter protein will be a fusion protein with the gene of interest and will be in-frame with the stop codon context element. In some embodiments, the reporter protein encoded by the gene may be a fluorescent protein or a fragment thereof, such as, e.g., GFP, RFP, YFP, or a functional derivative thereof. In some preferred though non-limiting embodiments, a reporter protein particularly suited for use with the present expression vector system may be β-galactosidase or a portion thereof, such as, e.g., the C-terminal portion of β-galactosidase, such as, e.g., the C-terminal 30 amino acids of β-galactosidase. In some embodiments, the portion of the gene encoding the reporter protein may include the nucleotide sequence GGTGGCGACGACTCCTGGAGCCCGTCAGTATCGGCGGAATTCCAGCTGAGCGCCGGT CGCTACCATTACCAGTTGGTCTGGTGTCAAAAATAA (SEQ ID NO: 15), or a functional equivalent thereof.

In certain non-limiting embodiments, methods for producing a recombinant protein in a host cell using an expression vector system as described herein may include the steps of obtaining an expression vector, the expression vector including a promoter capable of driving transcription in a host cell, a random ribosomal binding site (RRBS) functionally linked to and positioned downstream from the promoter, a stop codon context element comprising a stop codon and at least one 3-nucleotide codon in frame with the stop codon, and at least a portion of a gene encoding a reporter protein downstream of and in-frame with the stop codon context element. Further steps may include inserting a gene of interest into the expression vector between the RRBS and the stop codon context element, and introducing the expression vector into a host cell, and culturing the host cell under conditions permissive to said host cell expressing the recombinant protein.

In an embodiment, methods for producing a recombinant protein in a host cELL using an expression vector system as described herein may include inserting a gene of interest into the expression vector system so that the reading frame of the gene of interest is in-frame with the stop codon context element and the portion of the gene encoding a reporter protein. In some embodiments, the gene of interest that is inserted into the expression vector system, the stop codon context element, and the portion of the gene encoding the reporter protein form an open reading frame.

In an embodiment, the host cell that the expression vector system containing the gene of interest is inserted into is a eukaryotic cell. In alternate embodiments, the host cell that the expression vector system containing the gene of interest is inserted into is a prokaryotic cell, such as, e.g., an E. coli cell. The introduction of nucleic acids, including expression vectors, into eukaryotic and prokaryotic cells is a well-developed art and a variety of methods for doing so are well known to a fractioned possessing ordinary skill level in the art.

In an embodiment, the promoter of the expression vector system inserted into the host cell may be a eukaryotic promoter when the expression vector system is adapted for use in eukaryotic cells. Suitable eukaryotic promoters are known in the art and may include, though are not limited to, promoters such as CMV, MMTV, RSV and the like. Selecting a eukaryotic promoter for use in the present expression vector system is well within the purview of a practitioner having ordinary skill level in the art. In other embodiments, the promoter of the expression vector system inserted into the host cell may be a prokaryotic promoter when the expression vector system is adapted for use in prokaryotic cells. Suitable prokaryotic promoters are known in the art and may include, though are not limited to, promoters such as SP6, T7, T3 and P_{BAD} (araBAD) and the like. Selecting a prokaryotic promoter for use in the present expression vector system is well within the purview of a practitioner having ordinary skill level in the art.

In an embodiment, methods for producing a recombinant protein in a host cell using an expression vector system as described herein may include may include proving an expression vector having an RRBS that forms all or at least a portion of the 5′-UTR of an mRNA transcript transcribed from the expression vector. The RRBS may generally include the nucleotide sequence N₄R₆N_{X}, where N is A, T, G, or C, where R is A or G, and where X is an integer from 6 to 11 (SEQ ID NO: 1). In some non-limiting embodiments, an expression vector system may include the nucleotide sequence NNNNRRRRRRNNNNNN (SEQ ID NO: 2). In some embodiment, an RRBS may optionally include a translational initiation site. In other embodiments, a translational initiation site may be included in the user-provided gene of interest.

In some embodiments, methods for producing a recombinant protein in a host cell using an expression vector system as described herein may include optionally providing one or more cloning sites positioned between the RRBS and the stop codon context element. The cloning sites may be adapted or selected to facilitate the introduction of a user-provided or defined gene of interest into the expression vector. The optional cloning sites will preferably enable a user to introduce the gene of interest so that the gene forms an open reading frame with the stop codon context element and the portion of a gene encoding a reporter protein. In some embodiments the expression vector system may optionally be adapted for blunt-end cloning of a user-provided or defined cDNA between the RRBS and the stop codon context element. In other optional and non-limiting embodiments, an expression vector system may be adapted for TOPO®-cloning, for GATEWAY® Cloning or for TOPO® GATEWAY® cloning.

In some embodiments, methods for producing a recombinant protein in a host cell using an expression vector system as described herein may optionally include providing a nucleotide sequence encoding a Tag fusion protein. The nucleotide sequence encoding a Tag fusion protein may be in-frame with the stop codon context element and the portion of the gene encoding a reporter protein. Exemplary though non-limiting Tag-fusion proteins contemplated for use with the embodiments disclosed herein may include, though are not limited to, HIS₆ (SEQ ID NO: 3), HIS₈ (SEQ ID NO: 4), HIS₁₀ (SEQ ID NO: 5), MYC, FLAG, T7 Tag, GST, MBP, HA, S-Tag, V5 Epitope, Pel B, Xpress Epitope, NusA, CBP, GFP, Trx, Mistic, Sumo and DSCBc. In certain preferred though non-limiting embodiments, a Tag fusion protein may include one or more of HIS₆ (SEQ ID NO: 3), HIS₈ (SEQ ID NO: 4), HIS₁₀ (SEQ ID NO: 5), MYC, FLAG, GST, MBP and HA. Most preferably, a Tag fusion protein is one or more of HIS₆ (SEQ ID NO: 3), HIS₈ (SEQ ID NO: 4) and HIS₁₀ (SEQ ID NO: 5).

In an embodiment, methods for producing a recombinant protein in a host cell using an expression vector system as described herein may include providing an expression vector system having a stop codon context element. The stop codon context element may be selected such that up to about 10% of a protein, up to about 8% of a protein, up to about 5% of a protein, up to about 2% of a protein, up to about 1% of a protein, up to about 0.5% of a protein, up to about 0.2% of a protein, or up to about 0.1% of a protein translated from an mRNA produced in a cell using the expression vector system is expressed as a fusion protein with the reporter protein. In some embodiments, the stop codon context element may be selected such that between about 1% to about 10% of a protein, between about 0.5% to about 5% of a protein, between about 0.05% to about 0.5% of a protein, between about 0.01% to about 0.1% of a protein, or between about 0.01% to about 1% of a protein translated from an mRNA produced in a cell using the expression vector system is expressed as a fusion protein with the reporter protein.

In some embodiments, the nucleotide sequence of a stop codon context element for use in the embodiments described herein may be selected from the list consisting of the nucleotide sequences TAGNNN (SEQ ID NO: 6), TAANNN (SEQ ID NO: 7) or TGANNN (SEQ ID NO: 8), where N is A, T, G or C, and the nucleotide sequence NNN is selected to allow up to about 10%, up to about 5%, up to about 2%, up to about 1%, up to about 0.5%, up to about 0.2%, or up to about 0.1% or between about 1% to about 10%, between about 0.5% to about 5%, between about 0.05% to about 0.5%, between about 0.01% to about 0.1%, or between about 0.01% to about 1% suppression of the stop codon. In certain exemplary though non-limiting embodiments, the nucleotide sequence of a stop codon context element may be selected from the list of nucleotide sequences consisting of TAGNNN (SEQ ID NO: 9), TAANNN (SEQ ID NO: 10) or TGANNN (SEQ ID NO: 11), where N is A, T, G or C, and the nucleotide sequence NNN is selected from the list consisting of GAT, GCT, CGC, GTT, AAT, ACT, GAG, ATA, CAT, CGT, CCT, TAT, TCT, and ATT. In certain preferred though non-limiting embodiments, the nucleotide sequence of a stop codon context element may be selected from the list of nucleotide sequences consisting of TAGNNN (SEQ ID NO: 12), TAANNN (SEQ ID NO: 13) or TGANNN (SEQ ID NO: 14), where N is A, T, G or C, and the nucleotide sequence NNN is selected from either TAT or ATA.

In some embodiments, methods for producing a recombinant protein in a host cELL using an expression vector system as described herein may include providing an expression vector system having at least a portion of a gene encoding a reporter protein. The reporter protein will be a fusion protein with the gene of interest and will be in-frame with the stop codon context element. In some embodiments, the reporter protein encoded by the gene may be a fluorescent protein or a fragment thereof, such as, e.g., GFP, RFP, YFP, or a functional derivative thereof. In some preferred though non-limiting embodiments, a reporter protein particularly suited for use with the present expression vector system may be β-galactosidase or a portion thereof, such as, e.g., the C-terminal portion of β-galactosidase, such as, e.g., the C-terminal 30 amino acids of β-galactosidase. In some embodiments, the portion of the gene encoding the reporter protein may include the nucleotide sequence GGTGGCGACGACTCCTGGAGCCCGTCAGTATCGGCGGAATTCCAGCTGAGCGCCGGT CGCTACCATTACCAGTTGGTCTGGTGTCAAAAATAA (SEQ ID NO: 15), or a functional equivalent thereof.

An exemplary expression vector system in accordance with a preferred though non-limiting embodiment will be described in detail. It will be understood by those skilled in the art however, that the following description is merely for illustrative purposes only, and is not meant to unduly limit the scope of the invention solely to those specific embodiments described below. On the contrary, it will be readily apparent to those having ordinary skill level in the art that a variety of other embodiments not specifically described herein but which are within the purview of the skilled artisan without undue experimentation likewise fall within the scope of the present disclosure. Additionally, a number of elements described in the following embodiment may be considered optional only and are not necessarily required to practice the invention. Likewise, additional embodiments not specifically described below but which will readily apparent to those skilled in art without undue experimentation also fall within the spirit and scope of the invention.

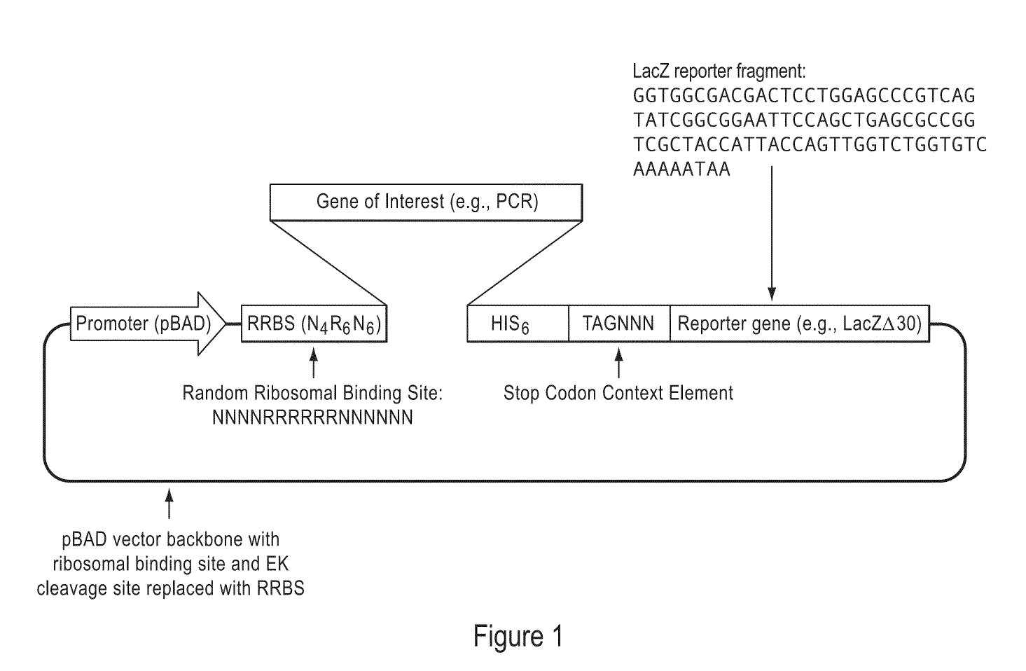

Turning to FIG. 1, an expression vector system in accordance with the embodiments described herein may include a vector backbone. Any suitable vector backbone can be used in the practice of the present invention without limitation. Optionally a eukaryotic expression vector may be used, although specific embodiments described herein are directed to prokaryotic expression vectors. Preferably, the vector used will be optimized for expression and/or propagation in a host cell. In certain exemplary though non-limiting, an expression vector system in accordance with the presently described embodiments may be derived from a commercially available expression vector, such as, e.g., the pBad expression vector available from Life Technologies. In some embodiments, a suitable vector backbone will include, in additional to sepcviofc elements described here, one or more additional elements commonly associated with expression vectors. For example, a vector backbone mat optionally include at least one selectable marker (not shown in FIG. 1), preferably more than one (e.g., Amp and/or Tet and/or Cam and/or araC). Optionally, a vector backbone may include at least one origin of replication (e.g., pBR322 ori). Optionally, the vector may contain a multi-cloning site. Alternatively, the vector may be optimized for Topo-cloning, blunt end cloning, Gateway® cloning, or any other cloning system optimized or otherwise recognized by those skilled in the art as being capable of allowing the insertion of a gene or a gene fragment into an expression vector for the purpose of transcribing a gene and translating a protein from an open reading frame.

Returning to FIG. 1, an expression vector system according to some embodiments may include a promoter suitable for driving expression of a gene of interest. In embodiments where a eukaryotic expression vector is used, a suitable eukaryotic promoter will be employed. A variety of eukaryotic promoters are known in the art, and any may be used in the practice of the invention without limitation. Strong, moderate or weak promoters are acceptable. In embodiments where a prokaryotic expression vector is used, a suitable bacterial promoter will be employed. A variety of prokaryotic promoters are known in the art, and any may be used in the practice of the invention without limitation. Exemplary bacterial promoters may include, e.g., T7, T3, SP6, P_{BAD} (araBAD) promoter etc.

In some preferred embodiments, an expression vector system as provided for herein may include a “random ribosomal binding site” (RRBS). In an embodiment, an expression vector system may be provided to a user as a vector library. Typically, the RRBS is a random region of variable length within the 5′UTR region of the gene of interest. The RRBS is designed to select clones that exhibit enhanced (or “optimized”) translation. Those clones having an RRBS sequence that promotes enhanced translation of the GOI will be selected based on color (or other) indicia that demonstrate elevated translation (e.g., higher levels of omega-complementation result in more intensely blue colonies. These will be selected as high expressers. Conversely, clones expressing low or intermediate amounts of protein may be selected if desired. The RRBS will generally contain at least the following elements: N₄R₆N_{X}; where N is A, T, G or C, R is a purine (A or G) and X is an integer from 6 to 11 (SEQ ID NO: 1). Optionally a translational start codon may be included at the 3′ end of the RRBS. Optionally, a translational start codon may be included immediately following the RRBS. Optionally, no start codon may be used as it may be provided as part of the user supplied Gene of Interest (GOI). The GOI may be prepared as a restriction fragment, a synthetic polynucleotide, a PCR fragment, or the like.

In some embodiments, an expression vector system may optionally contain a multiple cloning site. Optionally the vector may be adapted to facilitate blunt-end cloning, Topo-cloning, Gateway cloning, or the like. The expression vector will be adapted to allow a user to easily insert the GOI.

Downstream of the site in the vector where the GOI inserted, the vector may optionally contain a tag used for affinity purification, e.g., HIS₆ (SEQ ID NO: 3), MYC, FLAG etc. The tag will be in-frame with the GOI.

Downstream of the optional tag fusion (or the GOI if the tag is absent) is an in-frame stop codon (preferably amber stop TAG, but it could be any of the three stop codons) followed by a 3 nucleotide “context codon”. The context codon is selected to make “leakiness” more permissive and “fine-tuned”. Any of the three stop codons can be used, and using routine experimentation and optimized “context codon” can be found that permits the desired leakiness in the context of the stop codon and the strain of bacteria used. Context codons may be selected from the following High to Low GAT>GCT>CGC>GTT/AAT/ACT>GAG>ATA>CAT/CGT>CCT>TAT/TCT>ATT. TAT or ATA w selected for the commercial embodiment for low read-through. In the case of co-complementation ATA was used because it is more permissive. E. coli strains have some natural ability for read through, but selecting the proper context codon can fine-tune read-through ability. With TAGATA (SEQ ID NO: 16), TOP10, DH5c′ and DH10β seem to work well.

If-frame with the 2 codon “leaky” stop is a detectable reporter fusion. The fusion could be anything that is easily detectable in a culture or in colonies growing on plates. E.g., could be a fluorescent protein (GFR, RFP etc), lacZ or a mutant. In this case, we selected a short fusion of the C-terminal extremity of LacZ (namely LacZ Ct-term 30 amino acids that can ω-complement in E coli that are LacZ3′Δ. NB, the host cell must be able to express the reporter properly, especially in the case of 1acZCT. ω-complementation can only work in the appropriate host cell that has had endogenous or full-length LacZ knocked out and replaced with LacZAC. In this specific embodiment, the vector reporter fusion sequence is

(SEQ ID NO: 15)
GGTGGCGACGACTCCTGGAGCCCGTCAGTATCGGCGGAATTCCAGCTGAG
CGCCGGTCGCTACCATTACCAGTTGGTCTGGTGTCAAAAATAA

The following non-limiting examples are provided purely by way of illustration of exemplary embodiments, and in no way limit the scope and spirit of the present invention described above. Furthermore, it is to be understood that any inventions disclosed or claimed herein encompass all variations, combinations, and permutations of any one or more features described herein. Any one or more features may be explicitly excluded from the claims even if the specific exclusion is not set forth explicitly herein. It should also be understood that disclosure of a reagent for use in a method is intended to be synonymous with (and provide support for) that method involving the use of that reagent, according either to the specific methods disclosed herein, or other methods known in the art unless one of ordinary skill in the art would understand otherwise. In addition, where the specification and/or claims disclose a method, any one or more of the reagents disclosed herein may be used in the method, unless one of ordinary skill in the art would understand otherwise.

### EXAMPLES

### Example 1

### Plasmids

To construct the Random Ribsomal Element (RRE) vector library, a pBAD TOPO vector (Life Technologies) was re-circularized and digested with PME1 and BSA XI (New England Biolabs) to remove the ribosomal binding site, V5 epitope and TGA stop codon (Seq. 1). A gene of interest (CPm) insert with an XHO I restriction site, 6-Histidine tag (SEQ ID NO: 3), TAGATA sequence (SEQ ID NO: 16) and LacZ omega fragment (Seq. 2) at the C-terminal was cloned into Seq. 1 using GeneArt seamless cloning (Life Technologies). The resulting plasmid, pBAD-CPm E, was used as a template for generating the linear RRE vector library with XHO I and NCO I ends (Ylxn) using PCRwith pBAD XHO1 forward primer, RBS NCO 1 reverse primer (Table 1) and Phusion DNA polymerase (New England Biolabs). The resulting PCR product, Ylxn, was digested with NCO I and XHO I (New England Biolabs) and treated with Calf Intestinal Phosphatase (Life Technologies) to create the final linear library, Y1. To create the expression constructs, Y1CPm, CPm, aCPm insert with NCO I and XHO I ends (Seq. 3) was ligated into Y1 using ExpressLink T4 DNA ligase (Life Technologies). The complementation plasmid, LacZ A30, was generated by inserting an N-terminal fragment of Lac-Z (Seq. 4) between the NCO I, XHO I restriction sites in the pACYCDuet-i vector (Novagen).

### Transformation and Expression:

To screen for expression, Y1CPmand LacZ A30 were electroporated into a Top10 strain (Life Technologies). The transformation was spread on Luria broth (LB) agar plates supplemented with 100 ug/ml ampicillin (amp), 20 ug/mL chloramphenicol (cm), 0.1% arabinose and 40 ug/mL 5-bromo-4-chloro-3-indolyl-f3-D-galactopyranoside (X-gal) and grown overnight at 37° C. After 18 hours, dark blue colonies were picked and grown in 1 ml cultures of LB supplemented with 100 ug/ml ampicillin. For expression a 1:100 dilution of one of the LB cultures was used to inoculate a new 1 ml LB/100 ug/ml amp culture, which was grown to an A600 OD of 0.8 then induced with 0.02% arabinose overnight at 37° C.

### Ni-NTA Purification and Gel Analysis:

For purification of CPm, 100 ul of 10× FastBreak Cell Lysis Reagent (Promega)/Ni-NTA agarose (Life Technologies) slurry was added to the expression culture from above. The solution was incubated 1 hour at 4° C. on a circular rotator. The agarose beads were pelleted at 3,000×g for 1 minute, and the pellet washed twice with 1× phosphate buffered saline (PBS), pH 7.4. CPm protein was eluted from the beads with 300 mM imidazole in 1×PBS. Elution fractions were analyzed on a NuPAGE 4-12% Bis-Tris gel (Life Technologies) stained by Simply Blue Coomassie solution (Life Technologies).

TABLE 1
Primers

| Primer | |
|---|---|
| Name | Sequence |
| pBAD XHO1 | AAAAACTCGAGCATCATCATCATCATCATTAGATAGGT |
| forward | GGC (SEQ ID NO: 17) |
| RBS NCO 1 | AAAAACCATGGNNNNNNYYYYYYNNNAAACGGGTATGG |
| Reverse | AGAAACAGTA (SEQ ID NO: 18) |

Sequence of pBAD-TOPO BSA XI/PME I Digested Fragment

(SEQ ID NO: 19)
TAAACGGTCTCCAGCTTGGCTGTTTTGGCGGATGAGAGAAGATTTTCAGC
CTGATACAGATTAAATCAGAACGCAGAAGCGGTCTGATAAAACAGAATTT
GCCTGGCGGCAGTAGCGCGGTGGTCCCACCTGACCCCATGCCGAACTCAG
AAGTGAAACGCCGTAGCGCCGATGGTAGTGTGGGGTCTCCCCATGCGAGA
GTAGGGAACTGCCAGGCATCAAATAAAACGAAAGGCTCAGTCGAAAGACT
GGGCCTTTCGTTTTATCTGTTGTTTGTCGGTGAACGCTCTCCTGAGTAGG
ACAAATCCGCCGGGAGCGGATTTGAACGTTGCGAAGCAACGGCCCGGAGG
GTGGCGGGCAGGACGCCCGCCATAAACTGCCAGGCATCAAATTAAGCAGA
AGGCCATCCTGACGGATGGCCTTTTTGCGTTTCTACAAACTCTTTTTGTT
TATTTTTCTAAATACATTCAAATATGTATCCGCTCATGAGACAATAACCC
TGATAAATGCTTCAATAATATTGAAAAAGGAAGAGTATGAGTATTCAACA
TTTCCGTGTCGCCCTTATTCCCTTTTTTGCGGCATTTTGCCTTCCTGTTT
TTGCTCACCCAGAAACGCTGGTGAAAGTAAAAGATGCTGAAGATCAGTTG
GGTGCACGAGTGGGTTACATCGAACTGGATCTCAACAGCGGTAAGATCCT
TGAGAGTTTTCGCCCCGAAGAACGTTTTCCAATGATGAGCACTTTTAAAG
TTCTGCTATGTGGCGCGGTATTATCCCGTGTTGACGCCGGGCAAGAGCAA
CTCGGTCGCCGCATACACTATTCTCAGAATGACTTGGTTGAGTACTCACC
AGTCACAGAAAAGCATCTTACGGATGGCATGACAGTAAGAGAATTATGCA
GTGCTGCCATAACCATGAGTGATAACACTGCGGCCAACTTACTTCTGACA
ACGATCGGAGGACCGAAGGAGCTAACCGCTTTTTTGCACAACATGGGGGA
TCATGTAACTCGCCTTGATCGTTGGGAACCGGAGCTGAATGAAGCCATAC
CAAACGACGAGCGTGACACCACGATGCCTGTAGCAATGGCAACAACGTTG
CGCAAACTATTAACTGGCGAACTACTTACTCTAGCTTCCCGGCAACAATT
AATAGACTGGATGGAGGCGGATAAAGTTGCAGGACCACTTCTGCGCTCGG
CCCTTCCGGCTGGCTGGTTTATTGCTGATAAATCTGGAGCCGGTGAGCGT
GGGTCTCGCGGTATCATTGCAGCACTGGGGCCAGATGGTAAGCCCTCCCG
TATCGTAGTTATCTACACGACGGGGAGTCAGGCAACTATGGATGAACGAA
ATAGACAGATCGCTGAGATAGGTGCCTCACTGATTAAGCATTGGTAACTG
TCAGACCAAGTTTACTCATATATACTTTAGATTGATTTAAAACTTCATTT
TTAATTTAAAAGGATCTAGGTGAAGATCCTTTTTGATAATCTCATGACCA
AAATCCCTTAACGTGAGTTTTCGTTCCACTGAGCGTCAGACCCCGTAGAA
AAGATCAAAGGATCTTCTTGAGATCCTTTTTTTCTGCGCGTAATCTGCTG
CTTGCAAACAAAAAAACCACCGCTACCAGCGGTGGTTTGTTTGCCGGATC
AAGAGCTACCAACTCTTTTTCCGAAGGTAACTGGCTTCAGCAGAGCGCAG
ATACCAAATACTGTCCTTCTAGTGTAGCCGTAGTTAGGCCACCACTTCAA
GAACTCTGTAGCACCGCCTACATACCTCGCTCTGCTAATCCTGTTACCAG
TGGCTGCTGCCAGTGGCGATAAGTCGTGTCTTACCGGGTTGGACTCAAGA
CGATAGTTACCGGATAAGGCGCAGCGGTCGGGCTGAACGGGGGGTTCGTG
CACACAGCCCAGCTTGGAGCGAACGACCTACACCGAACTGAGATACCTAC
AGCGTGAGCTATGAGAAAGCGCCACGCTTCCCGAAGGGAGAAAGGCGGAC
AGGTATCCGGTAAGCGGCAGGGTCGGAACAGGAGAGCGCACGAGGGAGCT
TCCAGGGGGAAACGCCTGGTATCTTTATAGTCCTGTCGGGTTTCGCCACC
TCTGACTTGAGCGTCGATTTTTGTGATGCTCGTCAGGGGGGCGGAGCCTA
TGGAAAAACGCCAGCAACGCGGCCTTTTTACGGTTCCTGGCCTTTTGCTG
GCCTTTTGCTCACATGTTCTTTCCTGCGTTATCCCCTGATTCTGTGGATA
ACCGTATTACCGCCTTTGAGTGAGCTGATACCGCTCGCCGCAGCCGAACG
ACCGAGCGCAGCGAGTCAGTGAGCGAGGAAGCGGAAGAGCGCCTGATGCG
GTATTTTCTCCTTACGCATCTGTGCGGTATTTCACACCGCATATGGTGCA
CTCTCAGTACAATCTGCTCTGATGCCGCATAGTTAAGCCAGTATACACTC
CGCTATCGCTACGTGACTGGGTCATGGCTGCGCCCCGACACCCGCCAACA
CCCGCTGACGCGCCCTGACGGGCTTGTCTGCTCCCGGCATCCGCTTACAG
ACAAGCTGTGACCGTCTCCGGGAGCTGCATGTGTCAGAGGTTTTCACCGT
CATCACCGAAACGCGCGAGGCAGCAGATCAATTCGCGCGCGAAGGCGAAG
CGGCATGCATAATGTGCCTGTCAAATGGACGAAGCAGGGATTCTGCAAAC
CCTATGCTACTCCGTCAAGCCGTCAATTGTCTGATTCGTTACCAATTATG
ACAACTTGACGGCTACATCATTCACTTTTTCTTCACAACCGGCACGGAAC
TCGCTCGGGCTGGCCCCGGTGCATTTTTTAAATACCCGCGAGAAATAGAG
TTGATCGTCAAAACCAACATTGCGACCGACGGTGGCGATAGGCATCCGGG
TGGTGCTCAAAAGCAGCTTCGCCTGGCTGATACGTTGGTCCTCGCGCCAG
CTTAAGACGCTAATCCCTAACTGCTGGCGGAAAAGATGTGACAGACGCGA
CGGCGACAAGCAAACATGCTGTGCGACGCTGGCGATATCAAAATTGCTGT
CTGCCAGGTGATCGCTGATGTACTGACAAGCCTCGCGTACCCGATTATCC
ATCGGTGGATGGAGCGACTCGTTAATCGCTTCCATGCGCCGCAGTAACAA
TTGCTCAAGCAGATTTATCGCCAGCAGCTCCGAATAGCGCCCTTCCCCTT
GCCCGGCGTTAATGATTTGCCCAAACAGGTCGCTGAAATGCGGCTGGTGC
GCTTCATCCGGGCGAAAGAACCCCGTATTGGCAAATATTGACGGCCAGTT
AAGCCATTCATGCCAGTAGGCGCGCGGACGAAAGTAAACCCACTGGTGAT
ACCATTCGCGAGCCTCCGGATGACGACCGTAGTGATGAATCTCTCCTGGC
GGGAACAGCAAAATATCACCCGGTCGGCAAACAAATTCTCGTCCCTGATT
TTTCACCACCCCCTGACCGCGAATGGTGAGATTGAGAATATAACCTTTCA
TTCCCAGCGGTCGGTCGATAAAAAAATCGAGATAACCGTTGGCCTCAATC
GGCGTTAAACCCGCCACCAGATGGGCATTAAACGAGTATCCCGGCAGCAG
GGGATCATTTTGCGCTTCAGCCATACTTTTCATACTCCCGCCATTCAGAG
AAGAAACCAATTGTCCATATTGCATCAGACATTGCCGTCACTGCGTCTTT
TACTGGCTCTTCTCGCTAACCAAACCGGTAACCCCGCTTATTAAAAGCAT
TCTGTAACAAAGCGGGACCAAAGCCATGACAAAAACGCGTAACAAAAGTG
TCTATAATCACGGCAGAAAAGTCCACATTGATTATTTGCACGGCGTCACA
CTTTGCTATGCCATAGCATTTTTATCCATAAGATTAGCGGATCCTACCTG
ACGCTTTTTATCGCAACTCTCTACTGTTTCTCCATACCCGTTT

Sequence of CPm insert followed by Xhol, a 6× Histidine tag (SEQ ID NO: 3), TAGATA (SEQ ID NO: 16) and LacZ 3′ fragment—omega fragment.

(SEQ ID NO: 20)
ACTGTTTCTCCATACCCGTTTCAGGAGGAGAGGAGAGATATGAGTGTGAT
CGCTAAACAAATGACCTACAAGGTTTATATGTCAGGCACGGTCAATGGAC
ACTACTTTGAGGTCGAAGGCGATGGAAAAGGAAAGCCTTACGAGGGGGAG
CAGACGGTAAAGCTCACTGTCACCAAGGGCGGACCTCTGCCATTTGCTTG
GGATATTTTATCACCACAGTGTCAGTACGGAAGCATACCATTCACCAAGT
ACCCTGAAGACATCCCTGACTATGTGAAGCAGTCATTCCCGGAGGGCTAT
ACATGGGAGAGGATCATGAACTTTGAAGATGGTGCAGTGTGTACTGTCAG
CAATGATTCCAGCATCCAAGGCAACTGTTTCATCTACCATGTCAAGTTCT
CTGGTTTGAACTTTCCTCCCAATGGACCTGTCATGCAGAAGAAGACACAG
GGCTGGGAACCCAACACTGAGCGTCTCATTGCACGAGATGGAATGCTGCT
AGGAAACAACTTTATGGCTCTGAAGTTAGAAGGAGGCGGTCACTATTTGT
GTGAATTCAAAACTACTTACAAGGCAAAGAAGCCTGTGGAGATGCCAGGG
TATCACTATGTTGACCGCAAACTGGATGTAACCAATCACAACAAGGATTA
CACTTCGGTTGAGCTGTGTGAAATTTCCATTGCACGCAAACCTGTGGTCG
CCCTCGAGCATCACCACCATCACCATTAGATAGATAGCGACGACTCCTGG
AGCCCGTCAGTATCGGCGGAATTCCAGCTGAGCGCCGGTCGCTACCATTA
CCAGTTGGTCTGGTGTCAAAAATAATAA

### Sequence of Ncol/Spacer Followed by CPm and Xhol.

(SEQ ID NO: 21)
CCATGGGGAGTGTGATCGCTAAACAAATGACCTACAAGGTTTATATGTCA
GGCACGGTCAATGGACACTACTTTGAGGTCGAAGGCGATGGAAAAGGAAA
GCCTTACGAGGGGGAGCAGACGGTAAAGCTCACTGTCACCAAGGGCGGAC
CTCTGCCATTTGCTTGGGATATTTTATCACCACAGTGTCAGTACGGAAGC
ATACCATTCACCAAGTACCCTGAAGACATCCCTGACTATGTGAAGCAGTC
ATTCCCGGAGGGCTATACATGGGAGAGGATCATGAACTTTGAAGATGGTG
CAGTGTGTACTGTCAGCAATGATTCCAGCATCCAAGGCAACTGTTTCATC
TACCATGTCAAGTTCTCTGGTTTGAACTTTCCTCCCAATGGACCTGTCAT
GCAGAAGAAGACACAGGGCTGGGAACCCAACACTGAGCGTCTCATTGCAC
GAGATGGAATGCTGCTAGGAAACAACTTTATGGCTCTGAAGTTAGAAGGA
GGCGGTCACTATTTGTGTGAATTCAAAACTACTTACAAGGCAAAGAAGCC
TGTGGAGATGCCAGGGTATCACTATGTTGACCGCAAACTGGATGTAACCA
ATCACAACAAGGATTACACTTCGGTTGAGCTGTGTGAAATTTCCATTGCA
CGCAAACCTGTGGTCGCCCTCGAG

### Sequence of the N-Terminal Fragment of Lac-Z

(SEQ ID NO: 22)
ATGATAGATCCCGTCGTTTTACAACGTCGTGACTGGGAAAACCCTGGCGT
TACCCAACTTAATCGCCTTGCAGCACATCCCCCTTTCGCCAGCTGGCGTA
ATAGCGAAGAGGCCCGCACCGATCGCCCTTCCCAACAGTTGCGCAGCCTG
AATGGCGAATGGCGCTTTGCCTGGTTTCCGGTACCAGAAGCGGTGCCGGA
AAGCTGGCTGGAGTGCGATCTTCCTGAGGCCGATACTGTCGTCGTCCCCT
CAAACTGGCAGATGCACGGTTACGATGCGCCCATCTACACCAACGTAACC
TATCCCATTACGGTCAATCCGCCGTTTGTTCCCACGGAGAATCCGACGGG
TTGTTACTCGCTCACATTTAATGTTGATGAAAGCTGGCTACAGGAAGGCC
AGACGCGAATTATTTTTGATGGCGTTAACTCGGCGTTTCATCTGTGGTGC
AACGGGCGCTGGGTCGGTTACGGCCAGGACAGTCGTTTGCCGTCTGAATT
TGACCTGAGCGCATTTTTACGCGCCGGAGAAAACCGCCTCGCGGTGATGG
TGCTGCGTTGGAGTGACGGCAGTTATCTGGAAGATCAGGATATGTGGCGG
ATGAGCGGCATTTTCCGTGACGTCTCGTTGCTGCATAAACCGACTACACA
AATCAGCGATTTCCATGTTGCCACTCGCTTTAATGATGATTTCAGCCGCG
CTGTACTGGAGGCTGAAGTTCAGATGTGCGGCGAGTTGCGTGACTACCTA
CGGGTAACAGTTTCTTTATGGCAGGGTGAAACGCAGGTCGCCAGCGGCAC
CGCGCCTTTCGGCGGTGAAATTATCGATGAGCGTGGTGGTTATGCCGATC
GCGTCACACTACGTCTGAACGTCGAAAACCCGAAACTGTGGAGCGCCGAA
ATCCCGAATCTCTATCGTGCGGTGGTTGAACTGCACACCGCCGACGGCAC
GCTGATTGAAGCAGAAGCCTGCGATGTCGGTTTCCGCGAGGTGCGGATTG
AAAATGGTCTGCTGCTGCTGAACGGCAAGCCGTTGCTGATTCGAGGCGTT
AACCGTCACGAGCATCATCCTCTGCATGGTCAGGTCATGGATGAGCAGAC
GATGGTGCAGGATATCCTGCTGATGAAGCAGAACAACTTTAACGCCGTGC
GCTGTTCGCATTATCCGAACCATCCGCTGTGGTACACGCTGTGCGACCGC
TACGGCCTGTATGTGGTGGATGAAGCCAATATTGAAACCCACGGCATGGT
GCCAATGAATCGTCTGACCGATGATCCGCGCTGGCTACCGGCGATGAGCG
AACGCGTAACGCGAATGGTGCAGCGCGATCGTAATCACCCGAGTGTGATC
ATCTGGTCGCTGGGGAATGAATCAGGCCACGGCGCTAATCACGACGCGCT
GTATCGCTGGATCAAATCTGTCGATCCTTCCCGCCCGGTGCAGTATGAAG
GCGGCGGAGCCGACACCACGGCCACCGATATTATTTGCCCGATGTACGCG
CGCGTGGATGAAGACCAGCCCTTCCCGGCTGTGCCGAAATGGTCCATCAA
AAAATGGCTTTCGCTACCTGGAGAGACGCGCCCGCTGATCCTTTGCGAAT
ACGCCCACGCGATGGGTAACAGTCTTGGCGGTTTCGCTAAATACTGGCAG
GCGTTTCGTCAGTATCCCCGTTTACAGGGCGGCTTCGTCTGGGACTGGGT
GGATCAGTCGCTGATTAAATATGATGAAAACGGCAACCCGTGGTCGGCTT
ACGGCGGTGATTTTGGCGATACGCCGAACGATCGCCAGTTCTGTATGAAC
GGTCTGGTCTTTGCCGACCGCACGCCGCATCCAGCGCTGACGGAAGCAAA
ACACCAGCAGCAGTTTTTCCAGTTCCGTTTATCCGGGCAAACCATCGAAG
TGACCAGCGAATACCTGTTCCGTCATAGCGATAACGAGCTCCTGCACTGG
ATGGTGGCGCTGGATGGTAAGCCGCTGGCAAGCGGTGAAGTGCCTCTGGA
TGTCGCTCCACAAGGTAAACAGTTGATTGAACTGCCTGAACTACCGCAGC
CGGAGAGCGCCGGGCAACTCTGGCTCACAGTACGCGTAGTGCAACCGAAC
GCGACCGCATGGTCAGAAGCCGGGCACATCAGCGCCTGGCAGCAGTGGCG
TCTGGCGGAAAACCTCAGTGTGACGCTCCCCGCCGCGTCCCACGCCATCC
CGCATCTGACCACCAGCGAAATGGATTTTTGCATCGAGCTGGGTAATAAG
CGTTGGCAATTTAACCGCCAGTCAGGCTTTCTTTCACAGATGTGGATTGG
CGATAAAAAACAACTGCTGACGCCGCTGCGCGATCAGTTCACCCGTGCAC
CGCTGGATAACGACATTGGCGTAAGTGAAGCGACCCGCATTGACCCTAAC
GCCTGGGTCGAACGCTGGAAGGCGGCGGGCCATTACCAGGCCGAAGCAGC
GTTGTTGCAGTGCACGGCAGATACACTTGCTGATGCGGTGCTGATTACGA
CCGCTCACGCGTGGCAGCATCAGGGGAAAACCTTATTTATCAGCCGGAAA
ACCTACCGGATTGATGGTAGTGGTCAAATGGCGATTACCGTTGATGTTGA
AGTGGCGAGCGATACACCGCATCCGGCGCGGATTGGCCTGAACTGCCAGC
TGGCGCAGGTAGCAGAGCGGGTAAACTGGCTCGGATTAGGGCCGCAAGAA
AACTATCCCGACCGCCTTACTGCCGCCTGTTTTGACCGCTGGGATCTGCC
ATTGTCAGACATGTATACCCCGTACGTCTTCCCGAGCGAAAACGGTCTGC
GCTGCGGGACGCGCGAATTGAATTATGGCCCACACCAGTGGCGCGGCGAC
TTCCAGTTCAACATCAGCCGCTACAGTCAACAGCAACTGATGGAAACCAG
CCATCGCCATCTGCTGCACGCGGAAGAAGGCACATGGCTGAATATCGACG
GTTTCCATATGGGGATTGGTG

While certain preferred though non-limiting embodiments of the present invention have been and described and exemplified herein, it will be readily apparent to those having ordinary skill level in the art that such embodiments are provided by way of example only. Numerous variations, changes, and substitutions will now occur to those skilled in the art without departing from the spirit and the scope of the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. An expression vector comprising:
a promoter capable of driving transcription in a host cell;
a random ribosomal binding site (RRBS) functionally linked to and positioned downstream from the promoter;
a stop codon context element comprising a stop codon and at least onel 3-nucleotide codon in frame with the stop codon; and
at least a portion of a gene encoding a reporter protein downstream of and in-frame with the stop codon context element.

2. The expression vector according to claim 1, wherein the expression vector comprises a gene of interest inserted between the RRBS and the stop codon context element.

3. The expression vector according to claim 2, wherein the gene of interest is in-frame with the stop codon context element and the portion of the gene encoding a reporter protein.

4. The expression vector of claim 2, wherein the gene of interest, the stop codon context element and the portion of the gene encoding the reporter protein form an open reading frame.

5. The expression vector according to claim 3, therein the gene of interest forms an open reading frame with the stop codon contect element and the

[symbol]

6. The expression vector according to claim 1, wherein the expression vector is capable of driving the expression of a gene of interest in a host cell.

7. The expression vector according to claim 2, wherein the host cell is a eukaryotic cell.

8. The expression vector according to claim 2, wherein the host cell is a prokaryotic cell.

9. The expression vector according to claim 1, wherein the promoter is a eukaryotic promoter.

10. The expression vector according to claim 1, wherein the promoter is a prokaryotic promoter.

11. The expression vector according to claim 10, wherein the prokaryotic promoter is selected from the list consisting of SP6, T7, T3 and P_{BAD} (araBAD).

12. The expression vector according to claim 1, wherein the expression vector is adapted to accept a gene of interest cloned into the vector between the RRBS and the stop codon context element.

13. The expression vector according to claim 1, wherein the RRBS forms at least a portion of the 5′-UTR of an mRNA transcript transcribed from the expression vector.

14. The expression vector according to claim 1, wherein the RRBS comprises the nucleotide sequence N₄R₆N_{X}, where N is A, T, G, or C, where R is A or G, and where X is an integer from 6 to 11 (SEQ ID NO: 1).

15. The expression vector according to claim 14, wherein the RRBS comprises the nucleotide sequence NNNNRRRRRRNNNNNN (SEQ ID NO: 2).

16. The expression vector according to claim 1, wherein the RRBS comprises a translational initiation site.

17. The expression vector according to claim 2, wherein the gene of interest comprises a translational initiation site.

18. The expression vector according to claim 1, wherein the expression vector comprises one or more cloning sites positioned between the RRBS and the stop codon context element.

19. The expression vector according to claim 1, wherein the expression vector is adapted for blunt-end cloning of a cDNA between the RRBS and the stop codon context element.

20. The expression vector according to claim 1, wherein the expression vector is adapted for TOPO®-cloning.

21. The expression vector according to claim 1, wherein the expression vector is adapted for GATEWAY® Cloning.

22. The expression vector according to claim 1, wherein the expression vector comprises a nucleotide sequence encoding a Tag fusion protein.

23. The expression vector according to claim 22, wherein the nucleotide sequence encoding the Tag fusion protein is in-frame with the stop codon context element and the portion of the gene encoding a reporter protein.

24. The expression vector according to claim 22, wherein the tag fusion protein is selected from the list consisting of HIS₆ (SEQ ID NO: 3), HIS₈ (SEQ ID NO: 4), HIS₁₀ (SEQ ID NO: 5), MYC, FLAG, T7 Tag, GST, MBP, HA, S-Tag, V5 Epitope, Pel B, Xpress Epitope, NusA, CBP, GFP, Trx, Mistic, Sumo and DSCBc.

25. The expression vector according to claim 22, wherein the tag fusion protein is selected from the list consisting of HIS₆ (SEQ ID NO: 3), HIS₈ (SEQ ID NO: 4), HIS₁₀ (SEQ ID NO: 5), MYC, FLAG, GST, MBP and HA.

26. The expression vector according to claim 22, wherein the tag fusion protein is selected from the list consisting of HIS₆ (SEQ ID NO: 3), HIS₈ (SEQ ID NO: 4) and HIS₁₀ (SEQ ID NO: 5).

27. The expression vector according to claim 1, wherein the stop codon context element is selected such that up to about 10% of a protein expressed from the expression vector is expressed as a fusion protein with the reporter protein.

28. The expression vector according to claim 1, wherein the stop codon context element is selected such that up to about 8% of a protein expressed from the expression vector is expressed as a fusion protein with the reporter protein.

29. The expression vector according to claim 1, wherein the stop codon context element is selected such that up to about 5% of a protein expressed from the expression vector is expressed as a fusion protein with the reporter protein.

30. The expression vector according to claim 1, wherein the stop codon context element is selected such that up to about 2% of a protein expressed from the expression vector is expressed as a fusion protein with the reporter protein.

31. The expression vector according to claim 1, wherein the stop codon context element is selected such that up to about 1% of a protein expressed from the expression vector is expressed as a fusion protein with the reporter protein.

32. The expression vector according to claim 1, wherein the stop codon context element is selected such that up to about 0.5% of a protein expressed from the expression vector is expressed as a fusion protein with the reporter protein.

33. The expression vector according to claim 1, wherein the stop codon context element is selected such that up to about 0.2% of a protein expressed from the expression vector is expressed as a fusion protein with the reporter protein.

34. The expression vector according to claim 1, wherein the stop codon context element is selected such that up to about 0.1% of a protein expressed from the expression vector is expressed as a fusion protein with the reporter protein.

35. The expression vector according to claim 1, wherein the stop codon context element is selected such that between about 0.05% to about 0.5% of a protein expressed from the expression vector is expressed as a fusion protein with the reporter protein.

36. The expression vector according to claim 1, wherein the stop codon context element is selected such that between about 0.01% to about 0.1% of a protein expressed from the expression vector is expressed as a fusion protein with the reporter protein.

37. The expression vector according to claim 1, wherein the stop codon context element is selected such that between about 0.01% to about 1% of a protein expressed from the expression vector is expressed as a fusion protein with the reporter protein.

38. The expression vector according to claim 1, wherein the stop codon context element comprises the nucleotide sequence TAGNNN (SEQ ID NO: 6), TAANNN (SEQ ID NO: 7) or TGANNN (SEQ ID NO: 8), where N is A, T, G or C, and the nucleotide sequence NNN is selected to allow up to about 10% suppression of the stop codon.

39. The expression vector according to claim 1, wherein the stop codon context element comprises the nucleotide sequence TAGNNN (SEQ ID NO: 6), TAANNN (SEQ ID NO: 7) or TGANNN (SEQ ID NO: 8), where N is A, T, G or C, and the nucleotide sequence NNN is selected to allow up to about 5% suppression of the stop codon.

40. The expression vector according to claim 1, wherein the stop codon context element comprises the nucleotide sequence TAGNNN (SEQ ID NO: 6), TAANNN (SEQ ID NO: 7) or TGANNN (SEQ ID NO: 8), where N is A, T, G or C, and the nucleotide sequence NNN is selected to allow up to about 2% suppression of the stop codon.

41. The expression vector according to claim 1, wherein the stop codon context element comprises the nucleotide sequence TAGNNN (SEQ ID NO: 6), TAANNN (SEQ ID NO: 7) or TGANNN (SEQ ID NO: 8), where N is A, T, G or C, and the nucleotide sequence NNN is selected to allow up to about 1% suppression of the stop codon.

42. The expression vector according to claim 1, wherein the stop codon context element comprises the nucleotide sequence TAGNNN (SEQ ID NO: 6), TAANNN (SEQ ID NO: 7) or TGANNN (SEQ ID NO: 8), where N is A, T, G or C, and the nucleotide sequence NNN is selected to allow up to about 0.5% suppression of the stop codon.

43. The expression vector according to claim 1, wherein the stop codon context element comprises the nucleotide sequence TAGNNN (SEQ ID NO: 6), TAANNN (SEQ ID NO: 7) or TGANNN (SEQ ID NO: 8), where N is A, T, G or C, and the nucleotide sequence NNN is selected to allow up to about 0.1% suppression of the stop codon.

44. The expression vector according to claim 1, wherein the stop codon context element comprises the nucleotide sequence TAGNNN (SEQ ID NO: 9), TAANNN (SEQ ID NO: 10) or TGANNN (SEQ ID NO: 11), where N is A, T, G or C, and the nucleotide sequence NNN is selected from the list consisting of GAT, GCT, CGC, GTT, AAT, ACT, GAG, ATA, CAT, CGT, CCT, TAT, TCT, and ATT.

45. The expression vector according to claim 1, wherein the stop codon context element comprises the nucleotide sequence TAGNNN (SEQ ID NO: 12), TAANNN (SEQ ID NO: 13) or TGANNN (SEQ ID NO: 14), where N is A, T, G or C, and the nucleotide sequence NNN is selected from either TAT or ATA.

46. The expression vector according to claim 1, wherein the stop codon context element comprises the nucleotide sequence TAGATA (SEQ ID NO: 16).

47. The expression vector according to claim 1, wherein the stop codon context element comprises the nucleotide sequence TAGTAT (SEQ ID NO: 23).

48. The expression vector according to claim 1, wherein the gene encoding the reporter protein is a gene encoding at least a portion of a fluorescent protein.

49. The expression vector of claim 48, wherein the fluorescent protein is GFP, RFP, YFP, or a functional derivative thereof.

50. The expression vector according to claim 1, wherein the gene encoding the reporter protein is a gene encoding at least a portion of β-galactosidase.

51. The expression vector according to claim 1, wherein the gene encoding the reporter protein is a gene encoding the C-terminal portion of β-galactosidase.

52. The expression vector according to claim 1, wherein the gene encoding the reporter protein is a gene encoding the C-terminal 30 amino acids of β-galactosidase.

53. The expression vector according to claim 1, wherein the gene encoding the reporter protein comprises the nucleotide sequence GGGGCGACGACYCCYGGAGCCCGYCAGYAYCGGCGGAAYYCCAGCYGAGCG CCGGTCGCTACCATTACCAGTTGGTCTGGTGTCAAAAATAA (SEQ ID NO: 15), or a functional equivalent thereof.

54. A method for producing a recombinant protein in a host cell, said method comprising:
obtaining an expression vector comprising:
a promoter capable of driving transcription in a host cell;
a random ribosomal binding site (RRBS) functionally linked to and positioned downstream from the promoter;
a stop codon context element comprising a stop codon and at least one 3-nucleotide codon in frame with the stop codon; and
at least a portion of a gene encoding a reporter protein downstream of and in-frame with the stop codon context element;
inserting a gene of interest into the expression vector;
introducing the expression vector into a host cell; and
culturing the host cell under conditions permissive to said host cell expressing the recombinant protein.

55. The method according to claim 54, wherein said gene of interest is inserted between the RRBS and the stop codon context element.

56. The method according to claim 54, wherein said gene of interest is in-frame with the stop codon context element and the portion of the gene encoding a reporter protein.

57. The method according to claim 54, wherein the gene of interest, the stop codon context element and the portion of the gene encoding the reporter protein form an open reading frame.

58. The method according to claim 54, wherein the host cell is a eukaryotic cell.

59. The method according to claim 54, wherein the host cell is a prokaryotic cell.

60. The method according to claim 54, wherein the host cell is an E. coli cell.

61. The method according to claim 54, wherein the promoter is a eukaryotic promoter.

62. The method according to claim 54, wherein the promoter is a prokaryotic promoter.

63. The method according to claim 62, wherein the prokaryotic promoter is selected from the list consisting of SP6, T7, T3 and P_{BAD} (araBAD).

64. The method according to claim 54, wherein the RRBS forms at least a portion of the 5′-UTR of an mRNA transcript transcribed from the expression vector.

65. The method according to claim 54, wherein the RRBS comprises the nucleotide sequence N₄R₆N_{X}, where N is A, T, G, or C, where R is A or G, and where x is an integer from 6 to 11 (SEQ ID NO: 1).

66. The method according to claim 54, wherein the RRBS comprises the nucleotide sequence NNNNRRRRRRNNNNNN (SEQ ID NO: 2).

67. The method according to claim 54, wherein the RRBS comprises a translational initiation site.

68. The method according to claim 54, wherein the gene of interest comprises a translational initiation site.

69. The method according to claim 54, wherein the expression vector comprises one or more cloning sites positioned between the RRBS and the stop codon context element.

70. The method according to claim 54, wherein the expression vector is adapted for blunt-end cloning of a cDNA between the RRBS and the stop codon context element.

71. The method according to claim 54, wherein the expression vector is adapted for TOP010-cloning.

72. The method according to claim 54, wherein the expression vector is adapted for GATEWAY® Cloning.

73. The method according to claim 54, wherein the expression vector comprises a nucleotide sequence encoding a Tag fusion protein.

74. The method according to claim 73, wherein the nucleotide sequence encoding the Tag fusion protein is in-frame with the stop codon context element and the portion of the gene encoding a reporter protein.

75. The method according to claim 73, wherein the tag fusion protein is selected from the list consisting of HIS₆ (SEQ ID NO: 3), HIS₈ (SEQ ID NO: 4), HIS₁₀ (SEQ ID NO: 5), MYC, FLAG, T7 Tag, GST, MBP, HA, S-Tag, V5 Epitope, Pel B, Xpress Epitope, NusA, CBP, GFP, Trx, Mistic, Sumo and DSCBc.

76. The method according to claim 73, wherein the tag fusion protein is selected from the list consisting of HIS₆ (SEQ ID NO: 3), HIS₈ (SEQ ID NO: 4), HIS₁₀ (SEQ ID NO: 5), MYC, FLAG, GST, MBP and HA.

77. The method according to claim 73, wherein the tag fusion protein is selected from the list consisting of HIS₆ (SEQ ID NO: 3), HIS₈ (SEQ ID NO: 4) and HIS₁₀ (SEQ ID NO: 5).

78. The method according to claim 54, wherein the stop codon context element is selected such that up to about 10% of a protein expressed from the expression vector is expressed as a fusion protein with the reporter protein.

79. The method according to claim 54, wherein the stop codon context element is selected such that up to about 8% of a protein expressed from the expression vector is expressed as a fusion protein with the reporter protein.

80. The method according to claim 54, wherein the stop codon context element is selected such that up to about 5% of a protein expressed from the expression vector is expressed as a fusion protein with the reporter protein.

81. The method according to claim 54, wherein the stop codon context element is selected such that up to about 2% of a protein expressed from the expression vector is expressed as a fusion protein with the reporter protein.

82. The method according to claim 54, wherein the stop codon context element is selected such that up to about 1% of a protein expressed from the expression vector is expressed as a fusion protein with the reporter protein.

83. The method according to claim 54, wherein the stop codon context element is selected such that up to about 0.5% of a protein expressed from the expression vector is expressed as a fusion protein with the reporter protein.

84. The method according to claim 54, wherein the stop codon context element is selected such that up to about 0.2% of a protein expressed from the expression vector is expressed as a fusion protein with the reporter protein.

85. The method according to claim 54, wherein the stop codon context element is selected such that up to about 0.1% of a protein expressed from the expression vector is expressed as a fusion protein with the reporter protein.

86. The method according to claim 54, wherein the stop codon context element is selected such that between about 0.05% to about 0.5% of a protein expressed from the expression vector is expressed as a fusion protein with the reporter protein.

87. The method according to claim 54, wherein the stop codon context element is selected such that between about 0.01% to about 0.1% of a protein expressed from the expression vector is expressed as a fusion protein with the reporter protein.

88. The method according to claim 54, wherein the stop codon context element is selected such that between about 0.01% to about 1% of a protein expressed from the expression vector is expressed as a fusion protein with the reporter protein.

89. The method according to claim 54, wherein the stop codon context element comprises the nucleotide sequence TAGNNN (SEQ ID NO: 6), TAANNN (SEQ ID NO: 7) or TGANNN (SEQ ID NO: 8), where N is A, T, G or C, and the nucleotide sequence NNN is selected to allow up to about 10% suppression of the stop codon.

90. The method according to claim 54, wherein the stop codon context element comprises the nucleotide sequence TAGNNN (SEQ ID NO: 6), TAANNN (SEQ ID NO: 7) or TGANNN (SEQ ID NO: 8), where N is A, T, G or C, and the nucleotide sequence NNN is selected to allow up to about 5% suppression of the stop codon.

91. The method according to claim 54, wherein the stop codon context element comprises the nucleotide sequence TAGNNN (SEQ ID NO: 6), TAANNN (SEQ ID NO: 7) or TGANNN (SEQ ID NO: 8), where N is A, T, G or C, and the nucleotide sequence NNN is selected to allow up to about 2% suppression of the stop codon.

92. The method according to claim 54, wherein the stop codon context element comprises the nucleotide sequence TAGNNN (SEQ ID NO: 6), TAANNN (SEQ ID NO: 7) or TGANNN (SEQ ID NO: 8), where N is A, T, G or C, and the nucleotide sequence NNN is selected to allow up to about 1% suppression of the stop codon.

93. The method according to claim 54, wherein the stop codon context element comprises the nucleotide sequence TAGNNN (SEQ ID NO: 6), TAANNN (SEQ ID NO: 7) or TGANNN (SEQ ID NO: 8), where N is A, T, G or C, and the nucleotide sequence NNN is selected to allow up to about 0.5% suppression of the stop codon.

94. The method according to claim 54, wherein the stop codon context element comprises the nucleotide sequence TAGNNN (SEQ ID NO: 6), TAANNN (SEQ ID NO: 7) or TGANNN (SEQ ID NO: 8), where N is A, T, G or C, and the nucleotide sequence NNN is selected to allow up to about 0.1% suppression of the stop codon.

95. The method according to claim 54, wherein the stop codon context element comprises the nucleotide sequence TAGNNN (SEQ ID NO: 9), TAANNN (SEQ ID NO: 10) or TGANNN (SEQ ID NO: 11), where N is A, T, G or C, and the nucleotide sequence NNN is selected from the list consisting of GAT, GCT, CGC, GTT, AAT, ACT, GAG, ATA, CAT, CGT, CCT, TAT, TCT, and ATT.

96. The method according to claim 54, wherein the stop codon context element comprises the nucleotide sequence TAGNNN (SEQ ID NO: 12), TAANNN (SEQ ID NO: 13) or TGANNN (SEQ ID NO: 14), where N is A, T, G or C, and the nucleotide sequence NNN is selected from either TAT or ATA.

97. The method according to claim 54, wherein the stop codon context element comprises the nucleotide sequence TAGATA (SEQ ID NO: 16).

98. The method according to claim 54, wherein the stop codon context element comprises the nucleotide sequence TAGTAT (SEQ ID NO: 23).

99. The method according to claim 54, wherein the gene encoding the reporter protein is a gene encoding at least a portion of a fluorescent protein.

100. The method according to claim 99, wherein the fluorescent protein is GFP, RFP, YFP, or a functional derivative thereof.

101. The method according to claim 54, wherein the gene encoding the reporter protein is a gene encoding at least a portion of β-galactosidase.

102. The method according to claim 54, wherein the gene encoding the reporter protein is a gene encoding the C-terminal portion of β-galactosidase.

103. The method according to claim 54, wherein the gene encoding the reporter protein is a gene encoding the C-terminal 30 amino acids of β-galactosidase.

104. The method according to claim 54, wherein the gene encoding the reporter protein comprises the nucleotide sequence GGTGGCGACGACTCCTGGAGCCCGTCAGTATCGGCGGAATTCCAGCTGAGCG CCGGTCGCTACCATTACCAGTTGGTCTGGTGTCAAAAATAA (SEQ ID NO: 15), or a functional equivalent thereof.
